(19) United States
(12) Patent Application Publication
(10) Pub. No.: US 2012/0271258 A1
(43) Pub. Date: Oct. 25, 2012

(54) LEAK-PROOF SPUTUM SUCKER

(76) Inventor: Mei-Sheng Teng, Hong Kong (CN)

(21) Appl. No.: 13/535,299

(22) Filed: Jun. 27, 2012

Related U.S. Application Data
(63) Continuation-in-part of application No. 12/779,685, filed on May 13, 2010.

(51) Int. Cl. A61M 1/00

(52) U.S. Cl. 604/319

(57) ABSTRACT

The sputum sucker includes an outer tube and an inner axially slidably disposed in the outer tube. A normally-closed valve and a sleeve are disposed at a front end and a rear end of the outer tube, respectively. A bracket is fixed at a rear end of the inner tube. A connecting handle is articulatorily connected between the sleeve and the bracket. A torsion spring is disposed between the bracket and the connecting handle for normally exerting a force on the bracket to push the outer tube forwards. The normally-closed valve will seal up the inner tube when the connecting handle is not forced. The normally-closed valve will be broken open by the inner tube when the connecting handle is operated to pull the outer tube rearwards, and the normally-closed valve clings to the suction head to clean up while the normally-closed valve is being broken open and moved rearwards.

### CROSS-REFERENCE TO RELATED APPLICATIONS

This application is a continuation-in-part of U.S. patent application Ser. No. 12/779,685, filed May 13, 2010, now pending.

### BACKGROUND OF THE INVENTION

1. Field of the Invention

The present invention relates to suction devices, and more particularly to sputum suction tube for home use.

2. Description of the Prior Art

A suction tube works in such a way that the suction tube is inserted in the patient's respiratory tract and the suction pump is turned on to produce a negative pressure in the suction tube, sputum is sucked into the suction tube from the apertures in the distal end thereof, and then the respiratory tract is cleaned by pulling out the suction tube.

In real application, an operator may encounter a serious problem. When the negative pressure in the suction tube is insufficient or the negative pressure may suddenly disappear because of operational negligence, the sputum which has been sucked into the suction tube falls back into the respiratory tract by gravity or the patient's respiration. This may cause new respiratory tract obstruction. As a result, it may fail to clean the respiratory tract, or even make the patient worse. If the sputum flows or leaks out of the suction tube after being sucked thereinto, it will cause environmental contamination, and for a medial location with extremely high hygiene requirements, this is in no way different from a disaster.

### SUMMARY OF THE INVENTION

A primary object of the present invention is to provide a sputum sucker which can be automatically closed to prevent a leak of sputum when on force is exerted.

Another object of the present invention is to provide a sputum sucker which has an automatic cleaning function.

Still another object of the present invention is to provide a sputum sucker which is simply structured and easy-to-use.

To achieve the above object, the sputum sucker of the invention includes an outer tube and an inner axially slidably disposed in the outer tube. A normally-closed valve and a sleeve are disposed at a front end and a rear end of the outer tube, respectively. A bracket is fixed at a rear end of the inner tube. A connecting handle is articulatorily connected between the sleeve and the bracket. A torsion spring is disposed between the bracket and the connecting handle for normally exerting a force on the bracket to push the outer tube forwards. The normally-closed valve will seal up the inner tube when the connecting handle is not forced. The normally-closed valve will be broken open by the inner tube when the connecting handle is operated to pull the outer tube rearwards, and the normally-closed valve clings to the suction head to clean up while the normally-closed valve is being broken open and moved rearwards.

When in use, the sputum sucker is inserted into a patient's respiratory tract and the user presses the connecting handle and turns on a suction pump, pushing the outer tube move back, so that the front end of the inner tube pushes open the valve at the front end of the outer tube, and the sputum is sucked under the negative pressure from the respiratory tract into the suction head at the front end of the inner tube. The user can stop the sputum sucking operation at any time as he wants to simply by releasing the connecting handle. The torsion spring will push the outer tube forward to move back to its original position, and the valve at the front end of the outer tube will automatically be closed, the outer tube can firmly encloses the inner tube, preventing the occurrence of sputum leakage or backflow.

### BRIEF DESCRIPTION OF THE DRAWINGS

FIG. 1 is an exploded view of the present invention;

FIG. 2 is a side view of the present invention, showing that the sputum sucker is in a closed state;

FIG. 3 is a longitudinal sectional view of the present invention, showing that the leak proof sputum sucker is in a closed state.

FIG. 4 is a side view of the present invention, showing that the sputum sucker is in a sucking state; and

FIG. 5 is a longitudinal sectional view of the present invention, showing that the sputum sucker is in a sucking state.

### DETAILED DESCRIPTION OF THE PREFERRED EMBODIMENT

The present invention will be clearer from the following description when viewed together with the accompanying drawings, which show, for purpose of illustrations only, the preferred embodiment in accordance with the present invention.

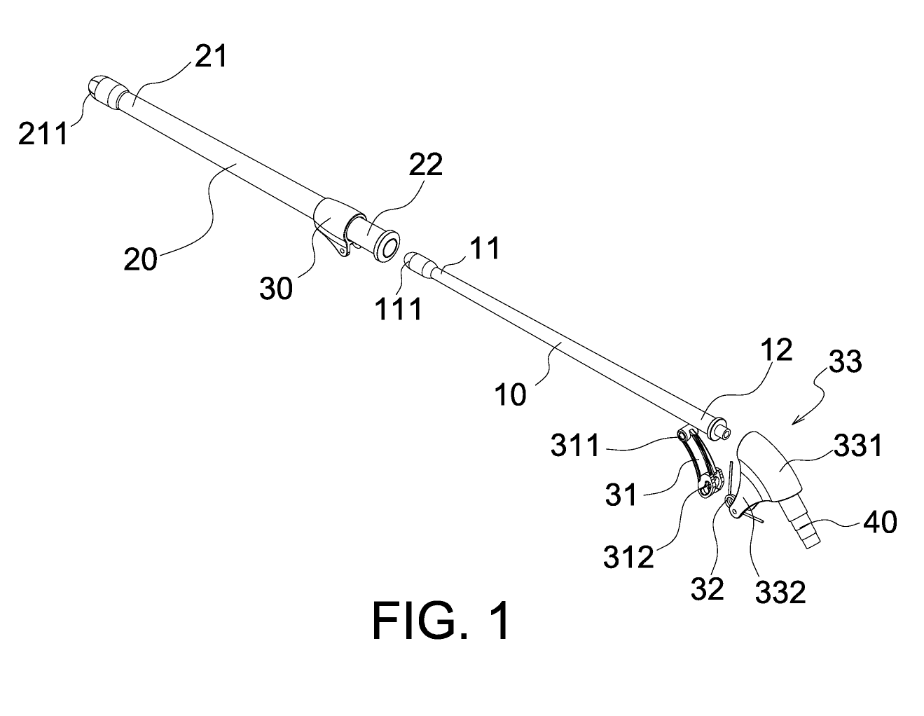

Referring to FIGS. 1-3, a leak-proof sputum sucker in accordance with the present invention comprises an inner tube 10, an outer tube 20, a sleeve 30, a connecting handle 31, a torsion spring 32 and a bracket 33. The outer tube 20 is straight and has a front end 21 and a rear end 22. A normally-closed valve 211 is disposed at the front end of the outer tube 20. The outer tube 20 is slidably telescoped on the inner tube 10. The inner tube 10 is straight and has a front end 11 and a rear end 12. The front end 11 of the inner tube 10 is formed with a suction head 111. The sleeve 30 is fastened to the rear end 22 of the outer tube 20.

The bracket 33 is composed of a tube portion 331 and a cradle portion 332. The tube portion 331 connects to and communicates with the inner tube 10. The inner tube 10 is further provided with a tube connector 40 for connecting an external tube (not shown). The connecting handle 31 is a bar with a first pivot hole 311 and a second pivot hole 312 separately at two ends thereof. The first pivot hole 311 is articulated to the sleeve 30 and the second pivot hole 312 is articulated to the cradle portion 332 of the bracket 33. Both ends of the torsion spring 32 are nipped between the connecting handle 31 and the bracket 33. Thus the torsion spring 32 can normally exerts a force to the connecting handle 31 against the bracket 33.

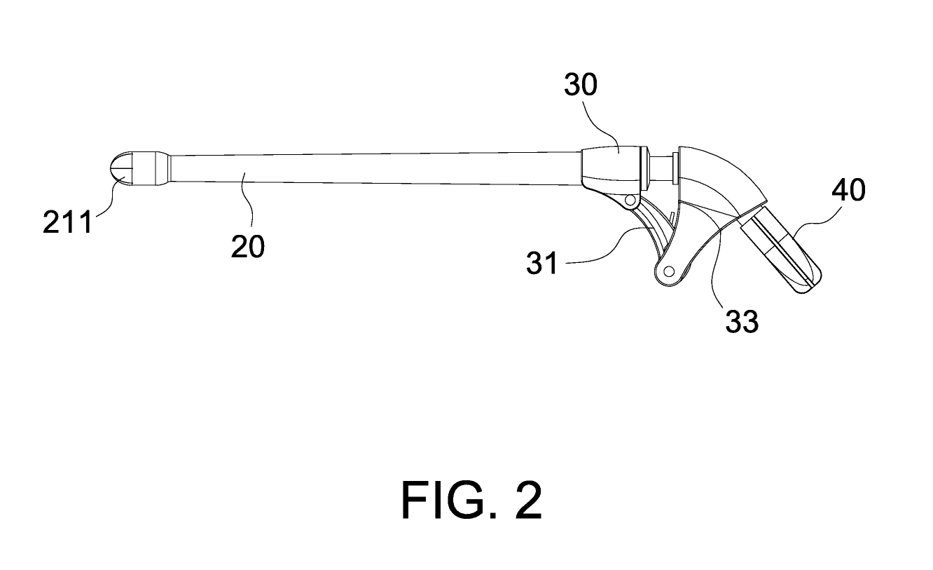

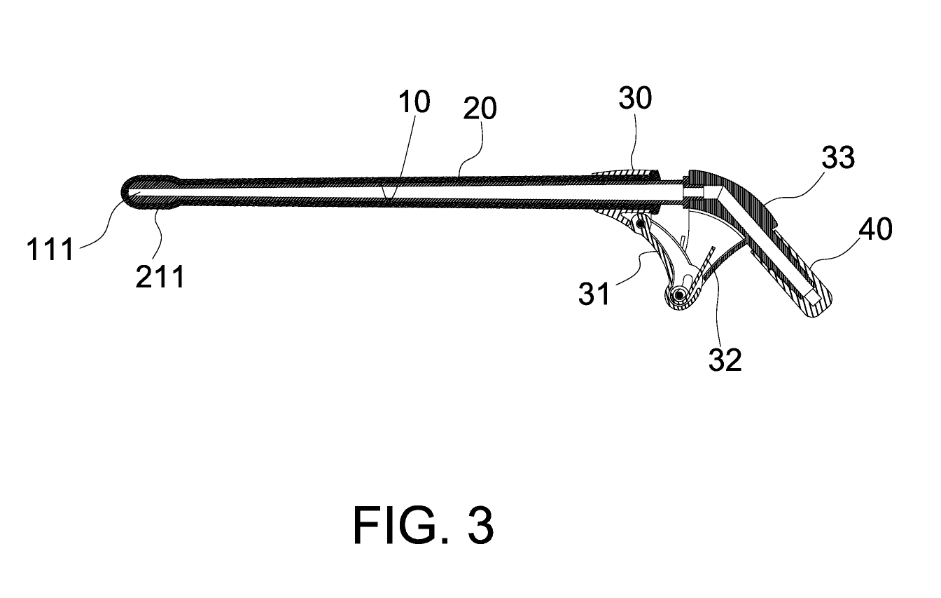

The normally-closed valve 211 is composed of five or six segments. It completely closes up when no force is exerted and may be outwards broken open by internal thrust. As shown in FIGS. 2 and 3, the outer tube 20 is normally pushed forwards by the torsion spring 32 through the connecting handle 31. Under a situation without any external force, the normally-closed valve 211 will automatically close up and seal up the suction head 111 of the inner tube 10.

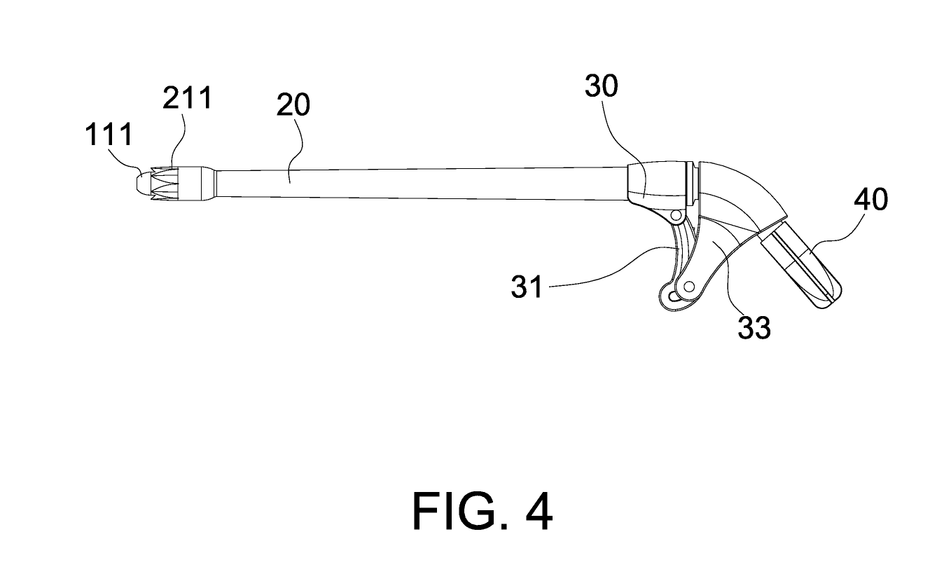

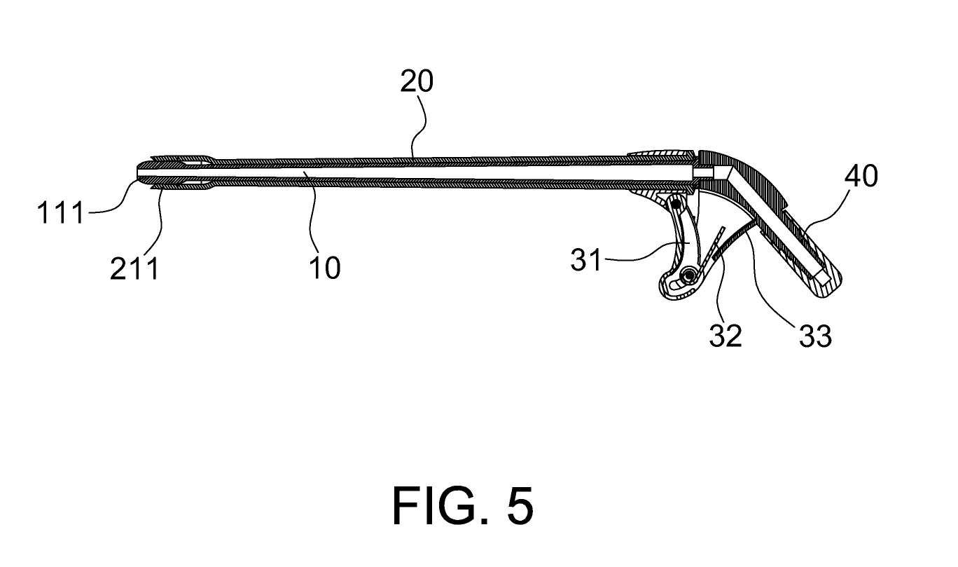

Please refer to FIGS. 4 and 5. When a user is using the sucker to suck sputum of a patient, the user presses the connecting handle 31, the outer tube 20 is pulled rearwards by the connecting handle 31 to move back and forth along the inner tube 10, so that the suction head 111 of the inner tube 10 will push open the normally-closed valve 211. And the sputum will be sucked out of the respiratory tract through the inner tube 10.

When sputum suction is done, the user releases the connecting handle 31, the torsion spring 32 will push the connecting handle 31 and the outer tube 20 forwards, and then the connecting handle 31 will push the sleeve 30 and the outer tube 20 to move forward along the inner tube 10. When the front end of the outer tube 20 moves over (to a position ahead of) the suction head 111 of the inner tube 10, the normally-closed valve 211 will automatically close up for preventing the occurrence of sputum leakage or backflow.

It should be noted that the normally-closed valve 211 clings to the suction head 111 while the normally-closed valve 211 is being broken open and moved rearwards because the normally-closed valve 211 has a shrinking force. This can clean up the suction head 111. In other words, every time the suction head 111 is exposed to be used, it has been cleaned up automatically.

While we have shown and described an embodiment in accordance with the present invention, it is clear to those skilled in the art that further embodiments may be made without departing from the scope of the present invention.

What is claimed is:

1. A sputum sucker comprising:
an outer tube, having a front end, a rear end and a normally-closed valve at the front end;
an inner tube, having a front end forming a suction head and a rear end, and axially slidably disposed in the outer tube;
a sleeve, fixed at the rear end of the outer tube;
a bracket, fixed at the rear end of the inner tube;
a connecting handle, articulatorily connected between the sleeve and the bracket; and
a torsion spring, disposed between the bracket and the connecting handle, and normally exerting a force on the bracket to push the outer tube forwards;
wherein the normally-closed valve will seal up the inner tube when the connecting handle is not forced, the normally-closed valve will be broken open by the inner tube when the connecting handle is operated to pull the outer tube rearwards, and the normally-closed valve clings to the suction head to clean up while the normally-closed valve is being broken open and moved rearwards.

2. The sputum sucker of claim 1, wherein the bracket is provided with a tube connector for connecting an external tube.

3. The sputum sucker of claim 1, wherein the bracket comprises a tube portion communicating with the inner tube and a cradle portion connecting with the connecting handle.

4. The sputum sucker of claim 2, wherein the bracket comprises a tube portion communicating with the inner tube and the tube connector and a cradle portion connecting with the connecting handle.

5. The sputum sucker of claim 1, wherein the connecting handle is a bar with a first pivot hole and a second pivot hole separately at two ends thereof, the first pivot hole and the second pivot hole are articulated to the sleeve and the cradle portion of the bracket, respectively.
